(19) United States
(12) Patent Application Publication
(10) Pub. No.: US 2014/0179963 A1
(43) Pub. Date: Jun. 26, 2014

(54) PROCESS AND SYSTEM FOR RECOVERING COMPONENTS FROM ALKYL BROMIDE SYNTHESIS

(71) Applicant: Marathon GTF Technology, Ltd., Houston, TX (US)

(72) Inventor: John J. Waycuilis, Cypress, TX (US)

(21) Appl. No.: 14/106,312

(22) Filed: Dec. 13, 2013

Related U.S. Application Data
(60) Provisional application No. 61/740,250, filed on Dec. 20, 2012.

(51) Int. Cl. C07C 7/00; C07C 17/357

(52) U.S. Cl. CPC C07C 7/005; C07C 17/357
USPC 570/254; 585/322; 585/310; 585/324; 422/187

(57) ABSTRACT

A process and system for recovering hydrogen bromide, methane, ethane and propane from butane and higher hydrocarbon products by means of condensation, cryogenic liquefaction and distillation, and for oxidation of the hydrogen bromide to bromine for re-use within a gas-conversion process for producing higher-molecular weight hydrocarbons.

### BACKGROUND OF THE INVENTION

The present invention relates generally to processes and systems for recovering substantially dry hydrogen bromide and other components from the effluent of alkyl bromide synthesis, and more particularly to processes and systems for separately recovering hydrogen bromide, methane (C₁), ethane (C₂) and propane (C₃) from butane and heavier (C₄₊) hydrocarbon products by means of condensation, cryogenic liquefaction and distillation, and for oxidation of the hydrogen bromide to bromine for re-use within a gas conversion process.

Natural gas, a fossil fuel, is primarily composed of methane and other light alkanes and has been discovered in large quantities throughout the world. When compared to other fossil fuels, natural gas is generally a cleaner energy source. For example, crude oil typically contains impurities, such as heavy metals, which are generally not found in natural gas. By way of further example, burning natural gas produces far less carbon dioxide than burning coal, per unit of heat energy released. However, challenges are associated with the use of natural gas in place of other fossil fuels. Many locations in which natural gas has been discovered are far away from populated regions and, thus, do not have significant pipeline structure and/or market demand for natural gas. Due to the low density of natural gas, the transportation thereof in gaseous form to more populated regions is expensive. Accordingly, practical and economic limitations exist to the distance over which natural gas may be transported in its gaseous form.

Cryogenic liquefaction of natural gas to form liquefied natural gas (often referred to as “LNG”) is often used to more economically transport natural gas over large distances. However, this LNG process is generally expensive, and there are limited regasification facilities in only a few countries for handling LNG. Converting natural gas to higher molecular weight hydrocarbons which, due to their higher density and value, are able to be more economically transported as a liquid can significantly expand the market for natural gas, particularly stranded natural gas produced far from populated regions. While a number of processes for the conversion of natural gas to higher molecular weight hydrocarbons have been developed, these processes have not gained widespread industry acceptance due to their limited commercial viability. Typically, these processes suffer from undesirable energy and/or carbon efficiencies that have limited their use.

One such process involves reacting lower molecular weight alkanes contained in a natural gas stream with bromine to produce alkyl bromides and hydrogen bromide. The resultant alkyl bromides may then be reacted over a suitable catalyst in the presence of hydrogen bromide to form olefins, higher molecular weight hydrocarbons or mixtures thereof as well as additional hydrogen bromide. As hydrogen bromide (HBr) is highly soluble in water or other aqueous solutions, hydrogen bromide is easily separated from less soluble substances (i.e. olefins and higher molecular hydrocarbons) by contact with an aqueous solution to form hydrobromic acid. However, hydrobromic acid solutions are highly corrosive to most metals, requiring expensive glass-lined or fluoropolymer-lined equipment. Furthermore, once water is introduced into such a process, a possibility exist of corrosion occurring in other non-lined portions of the process if condensation should occur in localized cold-spots created by inadequate insulation or heating, or due to, for example, process transients. Further, it would be advantageous and cost-effective if all component (C₁, C₂, HBr, C₃, and C₄₊) separations could be accomplished in concert to avoid significant loss of hydrocarbon products to HBr oxidation and to efficiently retain and recycle substantially all hydrogen bromide (HBr) and bromine (Br₂) within the process.

### BRIEF SUMMARY OF THE INVENTION

To achieve the foregoing and other objects, and in accordance with the purposes of the present invention, as embodied and broadly described herein, one embodiment of the present invention is a process comprising reacting a first quantity of bromine with lower molecular weight alkanes to form first bromination products comprising alkyl bromides. At least a portion of the alkyl bromides may be reacted in the presence of a catalyst to form an effluent containing unreacted lower molecular weight alkanes, hydrogen bromide and a C₄₊ hydrocarbon product comprising olefins, aromatics, higher molecular weight hydrocarbons, or mixtures thereof. At least a portion of the C₄₊ hydrocarbon product may be separated from the effluent by cooling said effluent to condense the at least a portion of the C₄₊ hydrocarbon product. The effluent from which at least a portion of the C4₊ hydrocarbon product has been separated is further cryogenically cooled to condense and separate C₂, HBr, C₃, and the remaining C₄₊ resulting in a residual vapor stream comprising primarily methane (C₁). The separated C₂, HBr, C₃, and C₄₊ is fractionated into a first stream containing predominately C₂, a second stream containing predominately HBr, and a third stream containing predominately C₃ and C₄₊.

Another embodiment of the present invention is a system comprising:

a bromination reactor for reacting bromine with lower molecular weight alkanes to form bromination products comprising alkyl bromides; a synthesis reactor for reacting at least a portion of said alkyl bromides in the presence of a catalyst to form an effluent containing unreacted lower molecular weight alkanes, hydrogen bromide and a C₄₊ hydrocarbon product comprising olefins, aromatics, higher molecular weight hydrocarbons, or mixtures thereof; a condenser for separating at least a portion of the C₄₊ hydrocarbon product from the effluent by cooling said effluent to condense said at least a portion of the C₄₊ hydrocarbon product; a cryogenic cooling unit for cooling the effluent from which said at least a portion of the C₄₊ hydrocarbon product has been separated to condense and separate C₂, HBr, C₃, and the remaining C₄₊ from the effluent resulting in a residual vapor stream comprising primarily methane (C1); and at least one fractionator for fractionating the separated C₂, HBr, C₃, and the remaining C₄₊ into separate streams containing predominately C₂, HBr and C₃ and C₄₊.

### BRIEF DESCRIPTION OF THE DRAWING

The accompanying drawing, which is incorporated in and forms a part of the specification, illustrates the embodiments of the present invention and, together with the description, serves to explain the principles of the invention.

FIG. 1 is a block flow diagram depicting an embodiment of the processes and systems of the present invention.

### DETAILED DESCRIPTION OF INVENTION

Gas streams that may be used as a feed stock for the methods described herein typically contain lower molecular weight alkanes. As utilized throughout this description, the term “lower molecular weight alkanes” refers to methane, ethane, propane, butane, pentane or mixtures of two or more of these individual alkanes. The lower molecular weight alkanes may be from any suitable source, for example, any source of gas that provides lower molecular weight alkanes, whether naturally occurring or synthetically produced. Examples of sources of lower molecular weight alkanes for use in the processes of the present invention include, but are not limited to, natural gas, coal-bed methane, re-gasified liquefied natural gas, gas derived from gas hydrates and/or clathrates, gas derived from anaerobic decomposition of organic matter or biomass, gas derived in the processing of tar sands, and synthetically produced natural gas or alkanes. Combinations of these may be suitable as well in some embodiments.

Suitable sources of bromine that may be used in various embodiments of the present invention include, but are not limited to, elemental bromine, bromine salts, aqueous hydrobromic acid, metal bromide salts, and the like. Combinations may be suitable, but as recognized by those skilled in the art, using multiple sources may present additional complications.

Certain embodiments of the processes and systems of the invention are described below. Although major aspects of what is believed to be the primary chemical reactions involved in the processes are discussed in detail as it is believed that they occur, it should be understood that side reactions may take place. One should not assume that the failure to discuss any particular side reaction herein means that that reaction does not occur. Conversely, those that are discussed should not be considered exhaustive or limiting. Additionally, although a FIGURE is provided that schematically shows certain aspects of the methods of the present invention, this FIGURE should not be viewed as limiting on any particular process of the invention.

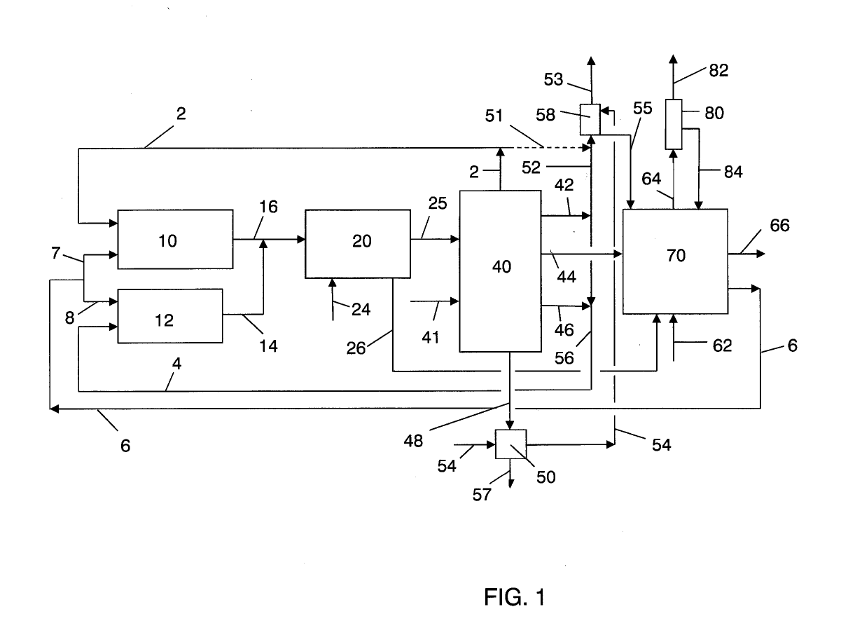

A block flow diagram generally depicting some aspects of certain embodiments of the processes and systems of the present invention is illustrated in FIG. 1. Gas stream 2 containing lower molecular weight alkanes may be pretreated to remove ethane and heavier (C₂₊) components as hereinafter described prior to being conveyed to at least one C₁₊, bromination reactor 10. The concentration of C₂₊ components in the feed gas stream 2 introduced into the C₁₊ bromination reactor 10 may be from about 0.1 mol % to about 10.0 mol %, more preferably from about 0.1 mol % to about 1.0 mol %, and most preferably from about 0.1 mol % to about 0.5 mol %. While some C₂₊ hydrocarbons may be tolerated in the C₁₊ bromination reactor 10 of FIG. 1, higher concentrations than set forth above may result in the rapid formation of carbon-containing coke-like solids which cause fouling and plugging in the bromination reactor as well as downstream components. Gas stream 2 may be combined with a dry bromine stream 7 prior to, upon introduction into or within the at least one C₁₊ bromination reactor 10. The ratio of methane to bromine that may be utilized in the feed to the at least one C₁₊ bromination reactor is a function of the C₂₊ content of the C₁₊ stream as well as the temperature. Lower C₂₊ content in the C₁₊ stream and operation at lower temperatures may allow operation at lower methane to bromine ratios.

Hence with the appropriate control of the C₂₊ content of the C₁₊ gas stream 2, the molar ratio of methane to bromine in the feed to the C₁₊ bromination reactor 10 is less than about 7 to 1 but greater than about 1.25 to 1, and preferably less than about 4 to 1 but greater than about 2 to 1, and more preferably less than or equal to about 3 to 1 but greater than about 2.5 to 1.

Further, in some embodiments, the dry bromine vapor in the mixture fed into the C₁₊ bromination reactor 10 may be substantially water-free. Applicant has discovered that, at least in some instances, this may be preferred because it appears that elimination of substantially all water vapor from the bromination step substantially eliminates the formation of unwanted carbon dioxide. This may increase the selectivity of alkane bromination to alkyl bromides, thus possibly eliminating the large amount of waste heat generated in the formation of carbon dioxide from alkanes.

In the C₁₊ bromination reactor 10, the lower molecular weight alkanes may be reacted exothermically with dry bromine vapor and the C₁₊ bromination reactor may preferably be operated at a pressure in the range of about 1 bar to about 80 bar, and more preferably about 1 bar to 30 bar, and at a temperature such that an outlet reaction temperature of about 470° C. to 530° C. is reached during a minimum residence time of about 60 seconds. As will be evident to a skilled artisan with the benefit of this disclosure, the bromination reaction in bromination reactor 10 may be an exothermic, homogeneous gas-phase reaction, a heterogeneous catalytic reaction, or a combination of both. Non-limiting examples of suitable catalysts that may be used in bromination reactor 10 include platinum, palladium, or supported non-stoichiometric metal oxy-halides, such as FeOₓBr_{y} or FeOₓCl_{y} or supported metal oxy-halides, such as TaOF₃, NbOF₃, ZrOF₂, SbOF₃ as described in Olah, et al., J. Am. Chem. Soc. 1985, 107, 7097-7105. It is believed that the upper limit of the operating temperature range may be greater than the upper limit of the reaction initiation temperature range to which the feed mixture is heated due to the exothermic nature of the bromination reaction. In the case of methane, it is believed that the formation of methyl bromide occurs in accordance with the following general overall reaction:

CH₄(g)+Br₂(g)→CH₃Br (g)+HBr(g)

Due to the free-radical mechanism of the homogeneous gas-phase bromination reaction, di-bromomethane and some tri-bromomethane and other alkyl bromides may also be formed. However, this reaction in accordance with the processes of the present invention often occurs with a relatively high degree of selectivity to methyl bromide due to the alkane-to-bromine ratio employed in bromination reactor 10 and the temperature and residence time for reaction. For example, in the case of the bromination of methane, a methane-to-bromine ratio of about 3:1 at a temperature of about 530° C. and residence time of about 60 seconds is believed to increase the selectivity to mono-halogenated methyl bromide to average approximately 88 to 90%. At these conditions, some di-bromomethane and only extremely small amounts of tri-bromomethane approaching the detectable limits also may be formed in the bromination reaction. If a lower methane-to-bromine ratio of approximately 2.6 to 1 and lower temperature of about 400° C. is utilized, selectivity to the mono-halogenated methyl bromide may fall to the range of approximately 65 to 75% depending residence time and other reaction conditions. At a methane-to-bromine ratio significantly less than about 2.5 to 1, unacceptable low selectivities to methyl bromide occurs, and, moreover, significant formation of undesirable di-bromomethane, tri-bromomethane, and carbon soot is observed. Higher alkanes, such as ethane, and the trace amounts of propane and butane, which may be present in the feed gas stream 2, may also be brominated, resulting in mono and multiple-brominated species such as ethyl bromides, propyl bromides and butyl bromides. However, because the higher alkanes have been found to be more reactive than methane, these tend to be preferentially reacted and become more poly-brominated at the higher temperature conditions in C₁₊ bromination reactor 10 that are required to brominate methane.

The residence time of the reactants in the C₁₊ bromination reactor(s) 10 necessary to achieve complete reaction of bromine may be relatively short and may be as little as 1-5 seconds under adiabatic reaction conditions. However, longer retention times of up to about 60 seconds have been found to improve the selectivity to mono-halogenated methyl bromide via a slower homogeneous, gas-phase reaction which occurs at the higher temperatures.

The C₁₊ bromination reactor(s) 10 may also contain a thermal or catalytic shift zone to facilitate this reaction. The temperature of the effluent from the thermal bromination zone that is fed to the thermal or catalytic shift zone may be in the range of about 350° C. to about 570° C., more preferably 500° C. to about 570° C., and most preferably 530° C. to about 570° C. As the C₁₊ thermal bromination reaction is exothermic, the feed gas and bromine introduced to the C₁₊ bromination reactor may be heated to a temperature within the about 300° C. to about 550° C. range to ensure that the effluent from the thermal bromination zone of the C₁₊ bromination reactor 64 is within the desired range for introduction into the thermal or catalytic shift zone given the reactor operating conditions of the thermal bromination reactor as will be evident to a skilled artisan. Alternatively, the effluent mixture from the thermal bromination zone or reactor may be heated or cooled to a temperature within the range of about 350° C. to about 570° C. prior to entry into the thermal or catalytic shift zone by any suitable means (not illustrated) as evident to a skilled artisan.

In the thermal or catalytic shift zone, a significant portion of the di-and tri- brominated alkanes that may be present in the alkyl bromides contained in the effluent from the thermal bromination zone may be selectively converted upon reaction with the unreacted alkane components, predominantly methane, present in the feed, to mono-brominated alkanes. As an example, where C₁ and di-bromomethane are the reactants, it is believed that the conversion occurs in accordance with the following general reaction:

CH₄+CH₂Br₂→2CH₃Br

Due to the high temperatures in the both the thermal and catalytic zones, elemental bromine may likely be essentially completely converted. The effluent from the thermal or catalytic shift zone of the C₁₊ bromination reactor which contains a significantly increased ratio of mono-brominated alkanes to di- or tri-brominated alkanes may then be transported to a at least one synthesis reactor 20. While the thermal and catalytic shift zones have been described above as contained within a single C₁₊ bromination reactor 10, these zones can each be contained in at least two separate reactors arranged in series as will be evident to a skilled artisan.

A gas stream 4 of C₂₊ components may be produced by the process or contained in the feed gas which are removed in stage 40 described below so that the excess C₂₊ and in particular C₃₊ may be separately processed in at least one C₂₊ thermal bromination reactor 12 together with a stream 8 of a suitable dry bromine feed. Gas stream 4 may be combined with a dry bromine stream 8 prior to, upon introduction into or within the at least one C₂₊ thermal bromination reactor 12. The C₂₊ thermal bromination reactor 12 operates at an alkane to bromine ratio of in the range of about 4 to 1 to about 1.25 to 1, and preferably in the range of about 2 to 1 to about 1.5 to 1 and at a temperature in the range of about 225° C. to 400° C.

A The higher alkanes, such as ethane, propane and butane will be brominated in the separate C2+ bromination reactor 12, resulting in mono- and multiple-brominated species such as ethyl bromides, propyl bromides and butyl bromides. The higher alkanes have been found to be more reactive than methane, requiring lower temperatures and lower residence times for complete reaction, and also a smaller excess of these higher alkanes is required to yield a high selectivity to mono-halogenated higher alkyl bromides, as compared to the bromination of methane.

The effluent stream 14 from the C₂₊ bromination reactor may be combined with the effluent stream 16 from the C₁₊ bromination reactor and the commingled effluent stream introduced into at least one synthesis reactor 20. This commingled effluent may be partially cooled by any suitable means, such as a heat exchanger (not illustrated), as will be evident to a skilled artisan before flowing to a synthesis reactor 20. The temperature to which the effluent is partially cooled is in the range of about 150° C. to about 420° C. when it is desired to convert the alkyl bromides to higher molecular weight hydrocarbons in synthesis reactor 20, or to range of about 150° C. to about 450° C. when it is desired to convert the alkyl bromides to olefins in synthesis reactor(s) 20. Synthesis reactor 20 is thought to oligomerize the alkyl units so as to form products that comprise olefins, higher molecular weight hydrocarbons or mixtures thereof. In synthesis reactor(s) 20, the alkyl bromides may be reacted exothermically at a temperature range of from about 150° C. to about 420° C., and a pressure in the range of about 1 to 80 bar, over a suitable catalyst to produce desired products (e.g., olefins, or aromatics and higher molecular weight hydrocarbons) and additional hydrogen bromide. Some temperature rise may occur in reactor(s) 20 due to the exothermic nature of the reaction.

The catalyst used in synthesis reactor(s) 20 may be any of a variety of suitable materials for catalyzing the conversion of the brominated alkanes to product hydrocarbons. In certain embodiments, this synthesis step may be carried out in fixed bed reactor synthesis reactor(s) 20 (which are alternatively taken off-line and periodically oxidatively regenerated) or may be carried out in moving-bed or fluidized-bed reactor(s) 20 (utilizing circulating solid catalyst particles which circulate between a reaction vessel and a regeneration vessel). A fluidized-bed or moving-bed of synthesis catalyst may also be used in certain circumstances, particularly in larger applications and may have certain advantages, such as constant removal of coke and a steady selectivity to product composition.

Examples of suitable catalysts for use in synthesis reactor(s) 20 include a fairly wide range of materials that have the common functionality of being acidic ion-exchangers and which also contain a synthetic crystalline alumino-silicate oxide framework. In certain embodiments, a portion of the aluminum in the crystalline alumino-silicate oxide framework may be substituted with magnesium, boron, gallium and/or titanium. In certain embodiments, a portion of the silicon in the crystalline alumino-silicate oxide framework may be optionally substituted with phosphorus. The crystalline alumino-silicate catalyst generally may have a significant anionic charge within the crystalline alumino-silicate oxide framework structure which may be balanced, for example, by cations of elements selected from the group H, Li, Na, K or Cs or the group Mg, Ca, Sr or Ba. Although zeolitic catalysts may be commonly obtained in a sodium form, a protonic or hydrogen form (via ion-exchange and subsequent calcining) is preferred, or a mixed protonic/sodium form may also be used. The zeolite may also be modified by ion exchange with other alkali metal cations, such as Li, K, or Cs, with alkali-earth metal cations, such as Mg, Ca, Sr, or Ba, or with transition metal cations, such as Ni, Mn, V, W. Such subsequent ion-exchange, may replace the charge-balancing counter-ions, but furthermore may also partially replace ions in the oxide framework resulting in a modification of the crystalline make-up and structure of the oxide framework. The crystalline alumino-silicate or substituted crystalline alumino-silicate may include a microporous or mesoporous crystalline aluminosilicate, but, in certain embodiments, may include a synthetic microporous crystalline zeolite, and, for example, being of the MFI structure such as ZSM-5. Moreover, the crystalline alumino-silicate or substituted crystalline alumino-silicate, in certain embodiments, may be subsequently impregnated with an aqueous solution of a Mg, Ca, Sr, or Ba salt. In certain embodiments, the salts may be a halide salt, such as a bromide salt, such as MgBr2. Optionally, the crystalline alumino-silicate or substituted crystalline alumino-silicate may also contain between about 0.1 to about 1 weight % Pt, about 0.1 to 5 weight % Pd, or about 0.1 to about 5 weight % Ni in the metallic state. Although, such materials are primarily initially crystalline, it should be noted that some crystalline catalysts may undergo some loss of crystallinity either due to initial ion-exchange or impregnation or chemical de-alumination treatments or due to operation at the reaction conditions or during regeneration and hence may also contain significant amorphous character, yet still retain significant, and in some cases improved activity and reduced selectivity to coke.

The particular catalyst used in synthesis reactor(s) 20 will depend, for example, upon the particular product hydrocarbons that are desired. For example, when product hydrocarbons having primarily C₃, C₄ and C₅₊ gasoline-range aromatic compounds and heavier hydrocarbon fractions are desired, a ZSM-5 zeolite catalyst may be used. When it is desired to produce product hydrocarbons comprising a mixture of olefins and C₅₊ products, an X-type or Y-type zeolite catalyst or SAPO zeolite catalyst may be used. Examples of suitable zeolites include an X-type, such as 10-X, or Y-type zeolite, although other zeolites with differing pore sizes and acidities may be used in embodiments of the present invention.

In addition to the catalyst, the temperature at which the synthesis reactor 20 is operated is an important parameter in determining the selectivity and conversion of the reaction to the particular product desired. For example, when an X-type, Y-type or SAPO zeolite catalyst is used and it is desired to produce olefins, it may be advisable to operate synthesis reactor 20 at a temperature within the range of about 250° C. to 500° C. Alternatively, in an embodiment involving a ZSM-5 zeolite catalyst operating in a slightly lower temperature range of about 150° C. to 420° C., cyclization reactions in the synthesis reactor occur such that the C₇₊ fractions contain primarily substituted aromatics and also light alkanes primarily in the C₃ to C₅₊ range. Surprisingly, very little ethane or C₂,-C₃ olefin components are found in the products in this case.

In some embodiments, the catalyst may be periodically regenerated in situ. One suitable method of regenerating the catalyst is to isolate reactor 20 from the normal process flow and purge it with an inert gas via line 24 at a pressure in a range from about 1 to about 5 bar at an elevated temperature in the range of about 400° C. to about 650° C. This should remove unreacted alkyl bromides and heavier hydrocarbon products adsorbed on the catalyst insofar as is practical. Optionally, the catalyst then may be subsequently oxidized by addition of air or inert gas-diluted air or oxygen to reactor 20 via line 24 at a pressure in the range of about 1 bar to about 30 bar at an elevated temperature in the range of about 400° C. to about 650° C. Carbon dioxide, unreacted alkyl bromides, heavier hydrocarbon products and residual air or inert gas may be removed from reactor 20 during the regeneration period via line 26 and processed in HBr oxidation stage 70 in a manner as hereinafter described.

The effluent from synthesis reactor(s) 20, which comprises unreacted lower molecular weight alkanes, hydrogen bromide and olefins, aromatics, higher molecular weight hydrocarbons or mixtures thereof, may be withdrawn from the synthesis reactor 20 via line 25 and transported to a C₄₊ separation, C₂₊ cryogenic liquefaction and C₂₊ component fractionation stage 40. Within this stage 40, the effluent from the synthesis reactor 20 may first be cooled to condense most of the C₄₊ hydrocarbon products and the remaining vapor fraction, containing most of the C₁, C₂, HBr, C₃ and some C₄, may be passed through a series of cryogenic cooling steps in which the C₂₊ components may be liquefied, and then may be fractionated via distillation, extractive distillation or both processes (using C₄, C₅ or other extraction agent). The resultant, purified C₂, HBr and C₃₊ streams may be removed from stage 40 via lines 42, 44 and 46, respectively. Feed gas may also be introduced via line 41 into the cryogenic liquefaction and fractionation stage, such that any C₂ and heavier components contained in the feed gas may also be simultaneously separated. Thus, C₁, C₂, HBr, C₃ and C₄₊ may be fractionated into separate streams in a series of distillation and/or extractive distillation steps within stage 40. The sequence of these distillation and fractionation steps may be arranged in various manners and operated at various pressures and heat integrated to varying degrees as indicated by the particular situation of energy and utility cost, capital cost and efficiency, as will be evident to an artisan skilled in process design. Regardless of the precise sequence and arrangement of the distillation and fractionation steps, the high-purity C₁ stream may be recycled to C₁₊ bromination step via line 2, a significant fraction 52 of the C₂ stream in line 42, and optionally a small fraction 51 of the C₁ stream in line 42, may be transported to contactor-separator 58. A C₄₊ product stream may be removed from stage 40 and transported to contactor-separator 50 via line 48. Water via line 54 may be used to wash HBr from the C₄₊ product stream 48 in contactor-separator 50 and from the C₁/C₂ stream in contactor-separator 58, respectively. The washed C₄₊ product stream may be removed from contactor-separator 50 via line 57 and the washed stream 53 containing C₂ and optionally also C₁ may be removed from contactor-separator 58 and utilized for fuel. The aqueous solution containing HBr may be conveyed from contactor-separator 58 via line 55 to thermal/catalytic HBr oxidation stage 70.

The balance of the C₂ stream in line 42 that is not utilized for fuel may be commingled with the C₃₊ stream (which may also contain some unconverted methyl bromide) in line 46, and transported via lines 56 and 4 to the C₂/C₃₊ bromination reactor(s) 12. Because C₂ is not as reactive with bromine as C₃ and C₄, it may be advantageous to satisfy the fuel demand of the process with as much C₂ as practical, thereby minimizing the amount of C₂ that is recycled to C₂₊ bromination reactor(s) 12, to avoid over-bromination of the C₃ and C₄ components. Alternatively, C₂ may be routed to a dedicated C₂ bromination reactor (not illustrated). The HBr stream (which may contain some small amounts of C₂ and or C₃) in line 44 may then be routed to a thermal/catalytic HBr oxidation stage 70 for conversion to elemental bromine and water. Since it is advantageous to keep trace water out of the hydrocarbon-containing portion of the process, a bromine-drying step may be included with the HBr oxidation stage 70 of the process.

Hydrogen bromide (HBr) in the absence of water has a liquid-vapor equilibrium curve intermediate to ethane and propane which permits the use of cryogenic liquefaction to recover ethane, HBr, propane and butanes from the methane-containing mixture present in the bromine-based process for the conversion of lower molecular weight alkanes into higher molecular-weight liquid hydrocarbon products. Further, distillation/extractive distillation technology for the separation of relatively pure component streams can be adapted to also separate a relatively pure stream of dry HBr from C₂ and C₃. It should be noted that some hydrogen bromide may be allowed in the C₂ and C₃ commingled stream recycled to the C₂₊ bromination reactor via lines 56 and 4, without significant negative impact thereby easing the difficulty of having to achieve a complete separation. Also some relatively small amounts of C₂ and C₃ contained in the HBr stream emanating from stage 40 via line 44 represent an acceptably minor loss. However, essentially complete HBr recovery from C₂ utilized as fuel for the process can be accomplished with a small amount of water washing in contactor-separator 58 yielding an essentially HBr-free fuel stream 53 for use in operating the process or for external use. The high-temperature thermal HBr oxidation step provides a “sink” for the small amount of aqueous hydrobromic acid resulting from washing of C₄₊ liquid products and the C₂/C₁ stream utilized as fuel.

In accordance with the oxidative regeneration of the synthesis catalyst as previously discussed, synthesis catalyst may be periodically-regenerated (in the case of fixed-bed synthesis reactors) or continuously-regenerated (in the case of moving-bed or fluidized-bed reactor systems) to remove heavy coke-like products that deactivate the catalyst in the course of the dehydrohalogenation/oligimerization reaction. Some amount of bromine may remain adsorbed on the catalyst as HBr or as organic bromides or brominated carbon, etc., and this is then liberated during the oxidative regeneration of the synthesis catalyst as HBr, but mostly as Br₂ contained in the regeneration off-gas. Because the HBr oxidation stage 70 employs a combination of high-temperature thermal oxidation, which operates in the range of about 950° C. to 1100° C., followed by catalytic oxidation, operated in the range of about 350° C. to 700° C. to essentially completely oxidize all the HBr in the feed to the oxidation system to bromine, such oxidation system provides a convenient “sink” for the synthesis catalyst regeneration off-gas which may contain bromine and may contain some residual oxygen and which is transported via line 26 to oxidation stage 70. Furthermore, because the HBr thermal oxidation step operates at high temperature and is a highly exothermic reaction, small amounts of aqueous acid resulting from the washing of the C₄₊ liquid products and C₂-containing fuel gas stream that may be transported to stage 70 via line 55 and may be sprayed into the high-temperature zone and vaporized. Any HBr contained in the vaporized acid, is then converted to Br₂ and recovered for re-use within the process. The initial hydrogen bromide-rich gas may be mixed with an oxidizing gas, transported to stage 70 via line 62 and heated within the thermal oxidation stage 70. Portions of the hydrogen bromide-rich gas are oxidized at high temperature in the thermal oxidation stage to produce elemental bromine and steam.

The unreacted remainder of the hydrogen bromide-rich gas and oxidizing gas is conveyed from the thermal oxidation stage to the catalytic oxidation stage where most or substantially all of the remaining unreacted hydrogen bromide-rich gas is oxidized in the presence of a catalyst to produce additional elemental bromine and steam. The resulting mixture of elemental bromine and steam is fed to a separation and product recovery step where the steam is condensed to water.

The resulting water and elemental bromine are separated and the elemental bromine is recovered as the end product via line 6, while water may be removed from HBr oxidation stage 70 via line 66.

A circulating regenerated aqueous liquid bromide stage 80 may be utilized to recover essentially all the bromine in the spent air stream leaving the oxidation stage 70 via line 64. In stage 80, bromine is absorbed from the spent air stream into an aqueous liquid stream comprising a bromide salt, and subsequently heating the liquid causes desorption of the bromine and regeneration of the liquid stream for re-use. The substantially pure spent air stream may be transported from stage 80 via line 82, while the recovered bromine may be transported to stage 70 via line 84.

In accordance with the embodiments of the present invention, the absence of water in the process stages 10, 12, 20 and 40 of the present invention permits the use of dry hydrogen bromide which is not particularly corrosive permits the use of relatively inexpensive carbon steel pressure vessels and stainless steel equipment therein. Further, hydrogen bromide (HBr) in the absence of water has a liquid-vapor equilibrium curve intermediate to ethane and propane which permits the use of cryogenic liquefaction to recover ethane, HBr, propane and butanes from the methane-containing mixture present in the bromine-based process for the conversion of lower molecular weight alkanes into higher molecular-weight liquid hydrocarbon products. Further, distillation/extractive distillation technology for the separation of relatively pure component streams can be adapted to also separate a relatively pure stream of dry HBr. While some hydrogen bromide may be allowed in the C₂ and C₃ commingled stream recycled to the C₂₊ bromination reactor without significant negative impact, easing the difficulty of achieving that separation. Also some relatively small amounts of C₂ and C₃ contained in the HBr stream emanating from stage 40 represents an acceptably minor loss. However, essentially complete HBr recovery from C₂ utilized as fuel for the process can be accomplished with a small amount of water washing. The high-temperature thermal HBr oxidation step provides a “sink” for the small amount of aqueous acid from washing of C₄₊ liquid products and the C₂ utilized as fuel.

Certain embodiments of the methods of the invention are described herein. Although major aspects of what is believed to be the primary chemical reactions involved in the methods are discussed in detail as it is believed that they occur, it should be understood that side reactions may take place. One should not assume that the failure to discuss any particular side reaction herein means that that reaction does not occur. Conversely, those that are discussed should not be considered exhaustive or limiting. Additionally, although figures are provided that schematically show certain aspects of the methods of the present invention, these figures should not be viewed as limiting on any particular method of the invention.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Although individual embodiments are discussed, the invention covers all combinations of all those embodiments. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. All numbers and ranges disclosed above may vary by some amount. Whenever a numerical range with a lower limit and an upper limit is disclosed, any number and any included range falling within the range are specifically disclosed.

What is claimed is:

1. A process comprising:
reacting a first quantity of bromine with lower molecular weight alkanes to form first bromination products comprising alkyl bromides;
reacting at least a portion of said alkyl bromides in the presence of a catalyst to form an effluent containing unreacted lower molecular weight alkanes, hydrogen bromide and a C₄₊ hydrocarbon product comprising olefins, aromatics, higher molecular weight hydrocarbons, or mixtures thereof;
separating at least a portion of the C₄₊ hydrocarbon product from the effluent by cooling said effluent to condense said at least a portion of the C₄₊ hydrocarbon product;
cryogenic cooling the effluent from which said at least a portion of the C₄₊ hydrocarbon product has been separated to condense and separate C₂, HBr, C₃ and the remaining C₄₊ hydrocarbon product thereby resulting in a C₁ residual vapor stream; and
fractionating the separated C₂, HBr, C₃ and the remaining C₄₊ hydrocarbon product from which said C₁ has been removed into a first stream containing predominately C₂, a second stream containing predominately HBr, and a third stream containing predominately C₃ and C₄₊.

2. The process of claim 1 further comprising:
transporting the C₁ that is removed from the effluent to the step of reacting.

3. The process of claim 1 further comprising:
contacting the condensed C₄₊ hydrocarbon product with water to remove residual hydrogen bromide therefrom.

4. The process of claim 3 further comprising contacting at least a portion of the first stream with said water to remove residual hydrogen bromide therefrom.

5. The process of claim 1 further comprising:
reacting a second quantity of bromine with the third stream, a first portion of the first stream or a combination thereof to form second bromination products comprising alkyl bromides.

6. The process of claim 5 wherein said second bromination products are combined with said first bromination products prior to said step of reacting.

7. The process of claim 1 further comprising;
thermally oxidizing at least a portion of the HBr contained in the second stream with an oxidizing gas at a first temperature sufficient to produce elemental bromine and steam.

8. The process of claim 7 wherein the first temperature is from about 950° C. to about 1100° C.

9. The process of claim 7 further comprising:
catalytically oxidizing substantially all of the remaining HBr contained in the second stream after thermal oxidation at a second temperature and in the presence of a catalyst and the oxidizing gas to produce additional elemental bromine and additional steam.

10. The process of claim 9 wherein the second temperature is from about 350° C. to about 700° C.

11. The process of claim 9 further comprising:
separating the water and the additional water from the elemental bromine and the additional elemental bromine.

12. The process of claim 11 further wherein the elemental bromine and the additional elemental bromine form at least a portion of the first quantity of bromine.

13. The process of claim 1 further comprising:
contacting at least a portion of the first stream with the water to remove residual hydrogen bromide therefrom.

14. The process of claim 13 further comprising:
thermally oxidizing at least a portion of the residual hydrogen bromide contained in the water with an oxidizing gas at a first temperature sufficient to produce elemental bromine and steam.

15. The process of claim 14 wherein the first temperature is from about 950° C. to about 1100° C.

16. The process of claim 9 further comprising:
absorbing bromine from the oxidizing gas into a liquid stream comprising a bromide salt; and
subsequently heating the liquid stream causing desorption of the bromine and regeneration of the liquid stream for re-use, said step of absorbing occurring after said steps of thermally and catalytically oxidizing.

17. The process of claim 1 a feed gas containing lower molecular weight alkanes is combined with the effluent prior to the steps of separating, cryogenic cooling and fractionating.

18. A system comprising:
a bromination reactor for reacting bromine with lower molecular weight alkanes to form bromination products comprising alkyl bromides;
a synthesis reactor for reacting at least a portion of said alkyl bromides in the presence of a catalyst to form an effluent containing unreacted lower molecular weight alkanes, hydrogen bromide and a C₄₊ hydrocarbon product comprising olefins, aromatics, higher molecular weight hydrocarbons, or mixtures thereof;
a condenser for separating at least a portion of the C₄₊ hydrocarbon product from the effluent by cooling said effluent to condense said at least a portion of the C₄₊ hydrocarbon product;
a cryogenic cooling unit for cooling the effluent from which said at least a portion of the C₄₊ hydrocarbon product has been separated to condense and separate C₂, HBr, C₃ and the remaining C₄₊ hydrocarbon product thereby resulting in a C₁ residual vapor stream; and
at least one fractionator for fractionating the separated C₂, HBr, C₃ and the remaining C₄₊ hydrocarbon product from which said C₁ has been removed into a first stream containing predominately C₂, a second stream containing predominately HBr, and a third stream containing predominately C₃ and C₄₊

19. The system of claim 18 further comprising:
an oxidizing unit for thermally and catalytically oxidizing hydrogen bromide with an oxidizing gas to produce elemental bromine and steam.

20. The system of claim 19 further comprising:
an absorber for removing bromine from the oxidizing gas into a liquid stream comprising a bromide salt.
